(19) United States
(12) Patent Application Publication
(10) Pub. No.: US 2020/0384451 A1
(43) Pub. Date: Dec. 10, 2020

(54) PHOSPHINE FREE COBALT BASED CATALYST, PROCESS FOR PREPARATION AND USE THEREOF

(71) Applicant: COUNCIL OF SCIENTIFIC AND INDUSTRIAL RESEARCH, New Delhi (IN)

(72) Inventors: Ekambaram BALARAMAN, Pune (IN); Siba Prasad MIDYA, Pune (IN); Vinod Gokulkrishna LANDGE, Pune (IN)

(21) Appl. No.: 16/644,850

(22) PCT Filed: Sep. 5, 2018
(86) PCT No.: PCT/IN2018/050572

§ 371 Date: Mar. 5, 2020

(30) Foreign Application Priority Data
Sep. 5, 2017 (IN) 201711031330

(51) Int. Cl. B01J 31/22; B01J 31/18; C07D 207/323; C07D 207/33; C07D 207/333; C07D 207/335; C07D 213/16; C07D 213/68; C07D 215/06; C07D 215/20; C07D 215/12; C07D 241/12

(52) U.S. Cl. CPC B01J 31/226; B01J 2231/349; C07D 207/323; C07D 207/33; C07D 207/333; C07D 207/335; C07D 213/16; C07D 213/68; C07D 215/06; C07D 215/20; C07D 215/12; C07D 241/12; B01J 2531/0205; B01J 2531/845; B01J 31/1805

(57) ABSTRACT

The present invention discloses a phosphine free cobalt based catalyst of formula (I) and a process for preparation thereof. The present invention further discloses a process for the synthesis of aromatic heterocyclic compounds of formula (II) and pyrazine derivative using the phosphine free cobalt based catalyst of formula (I).

### FIELD OF THE INVENTION

The present invention relates to a phosphine free cobalt based catalyst of formula (I) and a process for preparation thereof. The present invention further relates to phosphine free cobalt based catalyst of formula (I) useful for the preparation of aromatic heterocyclic compound of formula (II).

### BACKGROUND AND PRIOR ART OF THE INVENTION

The N-heterocyclic compounds have been highlighted as important scaffolds, as they have found applications in synthetic biology, pharmaceuticals, and material science. In particular, pyrrole constitutes one of the most important N-heterocyclic motifs and ubiquitous in natural products, drug intermediates, agrochemicals, dyes, and functional materials. Given their importance, the development of efficient strategies for the synthesis of pyrroles from simple feedstock chemicals is a prime focus in contemporary science. The classical approach to pyrrole synthesis involves the well-established Knorr, Paal-Knorr, and Hantzsch methods. Recently, metal-catalyzed inter- and intramolecular cyclization reactions have provided alternative approaches to access them. However, the direct and sustainable access to pyrroles under atom-economical, eco-benign conditions from simple alcohols is appealing, since alcohols can be derived from abundantly available lignocellulosic biomass by hydrogenolysis.

Transition-metal-catalyzed acceptor less dehydrogenation (AD) and hydrogen auto transfer (HA) reactions have been attracting much interest in recent times, in large part due to the excellent step-economy and high atom-efficiency. These strategies play a crucial role in activating the inert chemical bonds, such as the O—H bond of alcohols and the N—H bond of amines without pre-functionalization. In particular, catalytic acceptor less dehydrogenative coupling (ADC) reactions provide green synthetic methods for efficient organic transformations through tandem C—X (X═C, N, and O) bond-forming reactions with the liberation of H₂ and H₂O. Thus, ADC enables a direct and concise approach for the construction of diverse heterocyclic com-pounds from the easily available starting materials such as alcohols, amines, and unsaturated system. In 2013, Michlik and co-workers demonstrated the first direct synthesis of pyrroles from amino alcohols and secondary alcohols efficiently catalyzed by iridium (III)-complexes (Nature Chemistry; 2013, volume 5, pp 140-144).

Article titled “Direct synthesis of pyrroles by dehydrogenative coupling of β-aminoalcohols with secondary alcohols catalyzed by ruthenium pincer complexes” by D Srimani et al. published in Angew. Chem. Int. Ed.; 2013, 52, pp 4012-4015 reports synthesis of pyrroles in one step by using the acceptorless dehydrogenative coupling of amino alcohols with secondary alcohols (equivalent amounts), catalyzed by ruthenium pincer complexes (0.5 mol %) and a base (less than stoichiometric amounts) through selective C—N and C—C bond formation.

Article titled “Direct synthesis of pyridines and quinolines by coupling of γ-amino-alcohols with secondary alcohols liberating H₂ catalyzed by ruthenium pincer complexes” by D Srimani et al. published in Chem. Commun., 2013, 49, 6632-6634 reports a novel, one-step synthesis of substituted pyridine- and quinoline-derivatives was achieved by acceptorless dehydrogenative coupling of γ-aminoalcohols with secondary alcohols. The reaction involves consecutive C—N and C—C bond formation, catalyzed by a bipyridyl-based ruthenium pincer complex with a base.

Article titled “A sustainable catalytic pyrrole synthesis” by S Michlik et al. published in Nature Chemistry; 2013, volume 5, pp 140-144 reports a sustainable iridium-catalysed pyrrole synthesis in which secondary alcohols and amino alcohols are deoxygenated and linked selectively via the formation of C—N and C—C bonds. Two equivalents of hydrogen gas are eliminated in the course of the reaction, and alcohols based entirely on renewable resources can be used as starting materials. The catalytic synthesis protocol tolerates a large variety of functional groups, which includes olefins, chlorides, bromides, organometallic moieties, amines and hydroxyl groups.

Article titled “Manganese-catalyzed sustainable synthesis of pyrroles from alcohols and amino alcohols” by F Kallmeier et al. published in Angew. Chem. Int. Ed.; 2017, 56, pp 7261-7265 reports base-metal-catalyzed synthesis of pyrroles from alcohols and amino alcohols. The most efficient catalysts are Mn complexes stabilized by PN₅P ligands whereas related Fe and Co complexes are inactive. The reaction proceeds under mild conditions at catalyst loadings as low as 0.5 mol %, and has a broad scope and attractive functional-group tolerance. These findings may inspire others to use Mn catalysts to replace Jr or Ru complexes in challenging dehydrogenation reactions.

Article titled “Sustainable synthesis of quinolines and pyrimidines catalyzed by manganese PNP pincer complexes” by M Mastalir et al. published in J. Am. Chem. Soc., 2016, 138 (48), pp 15543-15546 reports an environmentally benign, sustainable, and practical synthesis of substituted quinolines and pyrimidines using combinations of 2-aminobenzyl alcohols and alcohols as well as benzamidine and two different alcohols, respectively. These reactions proceed with high atom efficiency via a sequence of dehydrogenation and condensation steps that give rise to selective C—C and C—N bond formations, thereby releasing 2 equiv of hydrogen and water. A hydride Mn(I) PNP pincer complex recently developed in our laboratory catalyzes this process in a very efficient way.

Article titled “A Ruthenium catalyst with unprecedented effectiveness for the coupling cyclization of γ-amino alcohols and secondary alcohols” by B Pan et al. published in ACS Catal., 2016, 6 (2), pp 1247-1253 reports a ruthenium catalyst for coupling cyclization of γ-amino alcohols and secondary alcohols. The ruthenium complex (8-(2-diphenylphosphinoethyl)aminotrihydroquinolinyl) (carbonyl)(hydrido) ruthenium chloride exhibited extremely high efficiency toward the coupling cyclization of γ-amino alcohols with secondary alcohols. The corresponding products, pyridine or quinoline derivatives, are obtained in good to high isolated yields. On comparison with literature catalysts whose noble-metal loading with respect to γ-amino alcohols reached 0.5-1.0 mol % for Ru and a record lowest of 0.04 mol % for Jr, the current catalyst achieves the same efficiency with a loading of 0.025 mol % for Ru.

Article titled “Regioselectively functionalized pyridines from sustainable resources” by S Michlik et al. published in Angew. Chem. Int. Ed., 2013, 52, pp 6326-6329 reports an Jr-catalyzed dehydrogenative condensation of alcohols and 1,3-amino alcohol used to construct pyridine derivatives regioselectively. This method provides access to unsymmetrically substituted pyridines and tolerates a wide variety of functional groups. Three equivalents of H₂ are generated per pyridine unit formed and the alcohol substrates become completely deoxygenated.

Importantly, it should be noted that all of the catalysts reported in the prior art for AD/HA reactions possess (electron-rich) phosphine ligands. Despite the tremendous success of phosphine ligands in homogeneous catalysis, they have encountered common drawbacks. For instance, their preparation is often non-trivial, requiring handling under an inert atmosphere, needing multi-step syntheses, etc. As a consequence, the phosphine ligands are expensive and can be challenging to make on a large scale, thereby hindering sustainable development. Therefore, there is need for an effective catalyst for the synthesis of aromatic heterocycles like pyrroles which will overcome drawbacks of phosphine based catalysts known in the prior art. Accordingly, the present invention provides a phosphine free cobalt based catalyst.

### OBJECTIVES OF THE INVENTION

The main objective of the present invention is to provide phosphine free cobalt based catalyst of formula (I).

Another objective of the present invention is to provide a process for the preparation of phosphine free cobalt based catalyst of formula (I).

Still another objective of the present invention is to provide a process for the preparation of aromatic heterocyclic compound of formula (II) by using phosphine free cobalt based catalyst of formula (I).

Yet another objective of the present invention is to provide a process for the preparation of pyrazine derivative by using phosphine free cobalt based catalyst of formula (I).

### SUMMARY OF THE INVENTION

Accordingly, present invention provides phosphine free cobalt based catalyst of formula (I)

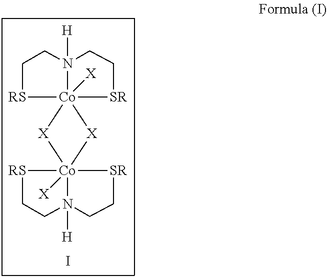

wherein,

R is selected from the group consisting of hydrogen, alkyl (linear or branched), substituted or unsubstituted aryl and heteroaryl containing O, N atoms;

X is selected from the group consisting of F, Cl, Br and I.

In an embodiment of the present invention, said phosphine free cobalt based catalyst of formula (I) is selected from cobalt based dimer complex of bis(2-(diethyl-λ3-sulfanyl)ethyl)amine, bis(2-(isopropylthio)ethyl)amine, bis(2-(phenylthio)ethyl)amine or bis(2-((substituted)phenylthio)ethyl)amine.

In yet another embodiment, present invention provides a process for the preparation of phosphine free cobalt based catalyst of formula (I) comprising the steps of:
i. preparing a solution of CoX₂ in solvent;
ii. preparing a solution of SNS ligand in solvent;
iii. mixing the solution of step (i) and (ii);
iv. stirring the reaction mixture of step (iii) at a temperature ranging from 25° C. to 30° C. for a time period ranging from 3 to 4 hours to yield cobalt based catalyst of formula (I).

In another embodiment of the present invention, said CoX₂ is selected from the group consisting of Cobalt (II) chloride (CoCl₂), Cobalt (II) bromide (CoBr₂) or Cobalt (II) Iodide (CoI₂).

In yet another embodiment of the present invention, said SNS ligand is selected from bis(2-(diethyl-λ3-sulfanyl)ethyl)amine (^{Et}SNS; L1) or bis(2-(isopropylthio)ethyl)amine (^{iosPr}SNS; L2).

In yet another embodiment of the present invention, said solvent is selected from the group consisting of methanol, ethanol, tetrahydrofuran, acetonitrile or diethylether.

In yet another embodiment, present invention provides a process for the synthesis of aromatic heterocyclic compound of formula (II)

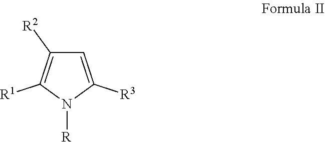

comprising heating a reaction mixture of amino alcohol, alcohol, catalyst of formula (I) and base in a ratio ranging between 1:2:0.2:1 to 1:0.5:0.25:1.5 and solvent at a temperature ranging from 150 to 180° C. for a time period ranging from 24 to 30 hours followed by cooling the reaction mixture to afford aromatic heterocyclic compound of formula (II).

In yet another embodiment of the present invention, said alcohol is selected from the group consisting of aliphatic short and long range primary alcohols, secondary alcohols, aromatic (substituted unsubstituted) primary and secondary alcohols, heteroaromatic alcohols or cyclic alcohols.

In yet another embodiment of the present invention, said alcohol is selected from the group consisting of 1-phenylethanol, 1-p-tolylethanol, 1-(4-chlorophenyl)ethanol, 1-(4-methoxyphenyl)ethanol, 1-(4-aminophenyl)ethanol, 1-(naphthalen-2-yl)ethanol, 1-(naphthalen-1-yl)ethanol, 2-decanol, 1-m-tolylethanol, 2-dodecanol, 1-(4-(trifluoromethyl)phenyl)ethanol and 1-(3-methoxyphenyl)ethanol.

In yet another embodiment of the present invention, said amino alcohol is selected from aliphatic and aromatic (β and γ) amino alcohols.

In yet another embodiment of the present invention, said amino alcohol is selected from the group consisting of 2-aminobutan-1-ol, 2-amino-3-methylbutan-1-ol, 2-amino-4-methylpentan-1-ol, 2-amino-3-methylpentan-1-ol, 2-amino-3-phenylpropan-1-ol, 2-amino-2-phenylethanol, 3-aminopropan-1-ol and (2-aminophenyl)methanol.

In yet another embodiment of the present invention, said base is selected from the group consisting of potasium tert-butoxide (t-BuOK), sodium tert-butoxide (t-BuONa), lithium tert-butoxide (t-BuOLi), potassium hydride (KH), sodium hydride (NaH), potassium Bis (trimethylsilyl) amide [KHMDS], lithium bis (trimethylsilyl) amide [LiHMDS], sodium isopropoxide (NaOiPr), sodium ethoxide (NaOEt) or sodium methoxide (NaOMe).

In yet another embodiment of the present invention, said solvent is selected from the group consisting of m-xylene, toluene, octane, mesitylene or decane.

In yet another embodiment of the present invention, said aromatic heterocyclic compound of formula (II) is selected from the group consisting of
i. 2-methyl-5-phenyl-1H-pyrrole (5a),
ii. 2-ethyl-5-phenyl-1H-pyrrole (5b),
iii. 2-isopropyl-5-phenyl-1H-pyrrole (5c),
iv. 2-isobutyl-5-phenyl-1H-pyrrole (5d),
v. 2-sec-butyl-5-phenyl-1H-pyrrole (5e),
vi. 2,5-diphenyl-1H-pyrrole (5f),
vii. 2-benzyl-5-phenyl-1H-pyrrole (5g),
viii. 2-isopropyl-5-p-tolyl-1H-pyrrole (5h),
ix. 2-(4-chlorophenyl)-5-isopropyl-1H-pyrrole (5i),
x. 2-isopropyl-5-(4-methoxyphenyl)-1H-pyrrole (5j),
xi. 4-(5-isopropyl-1H-pyrrol-2-yl)aniline (5k),
xii. 2-isopropyl-5-m-tolyl-1H-pyrrole (5l),
xiii. 2-isopropyl-5-(naphthalen-1-yl)-1H-pyrrole (5m),
xiv. 2-isopropyl-5-octyl-1H-pyrrole (5n),
xv. 2-isobutyl-5-(naphthalen-2-yl)-1H-pyrrole (5o),
xvi. 2-phenyl pyridine (7a),
xvii. 2-p-tolyl pyridine (7b),
xviii. 2-(4-methoxyphenyl) pyridine (7c),
xix. 2-m-tolylpyridine (7d),
xx. 2-octyl pyridine (7e),
xxi. 2-decyl pyridine (7f),
xxii. 2-phenyl quinoline (7g),
xxiii. 2-(3-methoxyphenyl) quinoline (7h),
xxiv. 2-(4-fluorophenyl)quinoline (7i),
xxv. 2-(4-(trifluoromethyl)phenyl)quinoline (7j) or
xxvi. 2-(naphthalen-2-yl)quinoline (7k).

In yet another embodiment, present invention provides a process for the synthesis of pyrazine derivative (C₄H₄N₂) comprises refluxing the reaction mixture of 1,2 amino alcohol and cobalt based catalyst of formula (I) as claimed in claim 1 in solvent at temperature in the range of 130 to 135° C. for the period in the range of 22 to 24 hrs under argon atmosphere to afford pyrazine derivative.

### BRIEF DESCRIPTION OF THE DRAWING

FIG. 1 represents X-ray crystal-structure analysis of phosphine free cobalt based catalyst of formula I with 50% probability of thermal ellipsoids.

FIG. 2 presents the process for the preparation of phosphine free cobalt based catalyst of formula (I).

FIG. 3 represents direct synthesis of 1H-pyrroles via dehydrogenative annulation reaction, wherein Reaction conditions are as follow: 0.25 mmol of 3, 0.5 mmol of 4, 2.5 mol % of catalyst 1, KOtBu (0.26 mmol), and 2 mL of m-xylene heated at reflux (150-180° C.) under argon atmosphere for 24 h.

FIG. 4 represents direct synthesis of pyridines and quinolines via dehydrogenative annulation reaction, wherein Reaction conditions are as follow: 0.25 mmol of 6, 0.5 mmol of 4, 2.5 mol % of catalyst 1, KOtBu (0.26 mmol), and 2 mL of m-xylene heated at reflux under argon atmosphere for 24 h.

FIG. 5 represents Synthesis of pyrazine (8) from β-aminoalcohol.

### DETAILED DESCRIPTION OF THE INVENTION

The present invention provides a phosphine free cobalt based catalyst of formula (I) and a process for the preparation thereof. The present invention further provides the base-metal (non-precious) catalyzed dehydrogenative annulation of γ-aminoalcohols and secondary alcohols into C2-substituted pyridine and quinoline derivatives via the acceptorless dehydrogenative coupling (ADC) strategy. The alcohols and γ-aminoalcohols are efficiently coupled via a sequence of acceptorless dehydrogenation and condensation to lead to the selective formation of C—N and C—C bonds. The acceptorless dehydrogenation leads to aromatization and the condensation step deoxygenates the alcohol component. Three equivalents of dihydrogen and water are liberated in the present reaction.

The present invention provides phosphine free cobalt based catalyst of formula (I)

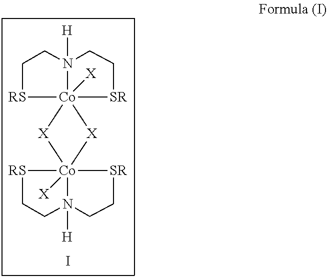

wherein

R is selected from the group consisting of hydrogen, alkyl (linear or branched), substituted or unsubstituted aryl and heteroaryl containing O, N atoms.

X is selected from group consisting of F, Cl, Br and I.

The phosphine free cobalt based catalyst of formula (I) is selected from the group consisting of cobalt based dimer complex of bis(2-(diethyl-λ3-sulfanyl)ethyl)amine, bis(2-(isopropylthio)ethyl)amine, bis(2-(phenylthio)ethyl)amine or bis(2-((substituted)phenylthio)ethyl)amine.

The phosphine free cobalt based catalyst of formula (I) is used for dehydrogenative annulation of unprotected amino alcohols with secondary alcohols for the direct synthesis of aromatic heterocyclic compound of formula (II) in presence of Transition-metal-catalyzed acceptorless dehydrogenation (AD) and hydrogen autotransfer (HA) reactions.

The present invention provides a process for the preparation of phosphine free cobalt based catalyst of formula (I) comprising
i. preparing a solution of CoX₂ in solvent
ii. preparing a solution of SNS ligand in solvent
iii. mixing the solution of step (i) and (ii)
iv. stirring the reaction mixture of step (iii) at a temperature ranging from 25° C. to 30° C. for a time period ranging from 3 to 4 hours to yield cobalt based catalyst of formula (I).

The SNS ligand is selected from bis(2-(diethyl-λ3-sulfanyl)ethyl)amine (^{Et}SNS; L1), and bis(2-(isopropylthio)ethyl)amine (^{iosPr}SNS; L2). The CoX₂ is selected from the group consisting of Cobalt (II) chloride (CoCl₂), Cobalt (II) bromide (CoBr₂) or Cobalt (II) Iodide (CoI₂). The solvent is selected from the group consisting of methanol, ethanol, tetrahydrofuran, acetonitrile or diethylether.

The process for the preparation of cobalt based catalyst of formula (I) is as depicted in FIG. 2.

The cobalt based catalyst 1 and 2 can be handled under an ordinary atmosphere (in air) as it is not sensitive towards moisture and oxygen over a considerable period of time (˜2 weeks). Complexes 1 and 2 catalyze the dehydrogenative coupling of unprotected 1,2- and 1,3-amino alcohols with secondary alcohols in an efficient manner that enables the direct and sustainable synthesis of 1H-pyrroles, and pyridines (or quinolines), respectively. This reaction involves the consecutive C—N and C—C bond formation with the liberation of hydrogen gas and water.

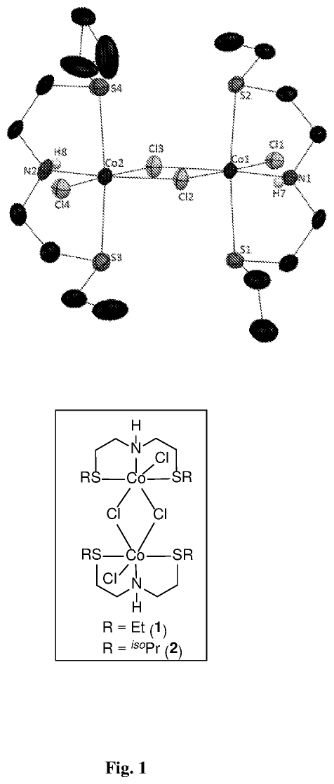

FIG. 1 depicts X-ray crystal-structure analysis of 1 with 50% probability of thermal ellipsoids. Hydrogen atoms (except N—H) are omitted for clarity. Selected bond length [Ao] and angle [o]: Co(1)-N(1) 2.124(7), Co(1)-S(1) 2.544(2), Co(1)-S(2) 2.555(2), Co(1)-Cl(1) 2.351(2), Co(1)-Cl(2) 2.548(2), Co(1)-Cl(3) 2.441(2), S(1)-Co(1)-S(2) 161.29(9), Cl(1)-Co(1)-Cl(2) 178.32(8), Cl(1)-Co(1)-N(1) 94.82(19), Co(1)-Cl(2)-Co(2) 95.74(8), S(1)-Co(1)-N(1) 81.58(18), S(2)-Co(1)-N(1) 82.39 (18).

The present invention also provides a process for the preparation of aromatic heterocyclic compound of formula (II) by using phosphine free cobalt based catalyst of formula (I) comprises heating the reaction mixture of amino alcohol, alcohol, catalyst of formula (I), base and solvent at the temperature ranging from 150−180° C. for the time period ranging from 24 to 30 hours followed by cooling the reaction mixture to afford aromatic heterocyclic compound of formula (II).

The aromatic heterocyclic compound of formula (II) is represented as follows:

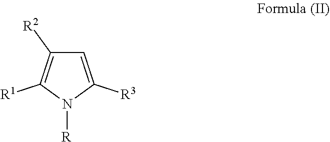

wherein;

n is selected from 0 or 1,

R is selected from the group consisting of hydrogen, alkyl (linear or branched), substituted or unsubstituted or aryl and heteroaryl contains 0, N atoms;

R¹, R², and R³ are same or different and independently selected from the group consisting of hydrogen, substituted or unsubstituted alkyl (linear or branched), substituted or unsubstituted aryl;

R¹ and R² may form a substituted or unsubstituted cyclic or heterocyclic ring.

The aromatic heterocyclic compounds of formula (II) is selected from the group consisting of 2-methyl-5-phenyl-1H-pyrrole (5a), 2-ethyl-5-phenyl-1H-pyrrole (5b), 2-isopropyl-5-phenyl-1H-pyrrole (5c), 2-isobutyl-5-phenyl-1H-pyrrole (5d), 2-sec-butyl-5-phenyl-1H-pyrrole (5e), 2,5-diphenyl-1H-pyrrole (5f), 2-benzyl-5-phenyl-1H-pyrrole (5g), 2-isopropyl-5-p-tolyl-1H-pyrrole (5h), 2-(4-chlorophenyl)-5-isopropyl-1H-pyrrole (5i), 2-isopropyl-5-(4-methoxyphenyl)-1H-pyrrole (5j), 4-(5-isopropyl-1H-pyrrol-2-yl)aniline (5k), 2-isopropyl-5-m-tolyl-1H-pyrrole (5l), 2-isopropyl-5-(naphthalen-1-yl)-1H-pyrrole (5m), 2-isopropyl-5-octyl-1H-pyrrole (5n), 2-isobutyl-5-(naphthalen-2-yl)-1H-pyrrole (5o), 2-phenyl pyridine (7a), 2-p-tolyl pyridine (7b), 2-(4-methoxyphenyl) pyridine (7c), 2-m-tolylpyridine (7d), 2-octyl pyridine (7e), 2-decyl pyridine (7f), 2-phenyl quinoline (7g), 2-(3-methoxyphenyl) quinoline (7h), 2-(4-fluorophenyl)quinoline (7i), 2-(4-(trifluoromethyl)phenyl)quinoline (7j) or 2-(naphthalen-2-yl)quinoline (7k).

The alcohol is selected from the group consisting of aliphatic short and long range primary alcohols, secondary alcohols, substituted or unsubstituted aromatic primary alcohols, aromatic secondary alcohols, heteroaromatic alcohols or cyclic alcohols. Preferably, the alcohol is selected from the group consisting of 1-phenylethanol, 1-p-tolylethanol, 1-(4-chlorophenyl)ethanol, 1-(4-methoxyphenyl)ethanol, 1-(4-aminophenyl)ethanol, 1-(naphthalen-2-yl)ethanol, 1-(naphthalen-1-yl)ethanol, 2-decanol, 1-m-tolylethanol, 2-dodecanol, 1-(4-(trifluoromethyl)phenyl)ethanol and 1-(3-methoxyphenyl)ethanol.

The amino alcohol is selected from aliphatic and aromatic (β and γ) amino alcohols. Preferably, the amino alcohol is selected from the group consisting of 2-aminobutan-1-ol, 2-amino-3-methylbutan-1-ol, 2-amino-4-methylpentan-1-ol, 2-amino-3-methylpentan-1-ol, 2-amino-3-phenylpropan-1-ol, 2-amino-2-phenylethanol, 3-aminopropan-1-ol and (2-aminophenyl)methanol.

The solvent is selected from the group consisting of m-xylene, Toluene, Octane, mesitylene or Decane.

The base is selected from the group consisting of potasium tert-butoxide (t-BuOK), sodium tert-butoxide (t-BuONa), lithium tert-butoxide (t-BuOLi), potassium hydride (KH), sodium hydride (NaH), potassium Bis (trimethylsilyl) amide [KHMDS], Lithium bis (trimethylsilyl) amide [LiHMDS], Sodium isopropoxide (NaOiPr), Sodium ethoxide (NaOEt) or Sodium methoxide (NaOMe).

The phosphine free cobalt based catalyst of formula (I) is selected from the group consisting of cobalt based dimer complex of bis(2-(diethyl-λ3-sulfanyl)ethyl)amine, bis(2-(isopropylthio)ethyl)amine, bis(2-(phenylthio)ethyl)amine, and bis(2-((substituted)phenylthio)ethyl)amine.

The present invention further provides direct synthesis of 1H-pyrroles via dehydrogenative annulation reaction as depicted in FIG. 3.

In the dehydrogenative condensation steps, two equivalents of H₂ are liberated per pyrrole motif, thus making the protocol completely environmentally benign. The reaction proceeded successfully with both aliphatic and aromatic unprotected β-aminoalcohols and gave the de-sired 1H-pyrroles in moderate to good yields (58%-86%).

The present invention also provides direct synthesis of pyridines and quinolines via dehydrogenative annulation reaction as shown in FIG. 4.

All of the pyridine derivatives (7a-f) are isolated in moderate to good yields (60-83%). C-2 substituted quinolines (7g-k) are also prepared involving dehydrogenative cyclization of 2-aminobenzyl alcohol with various secondary alcohols using our established protocol in very good yields (up to 87%). Thus, the phosphine-free cobalt (II) catalyst as disclosed in the present invention displayed remarkable activity in the sustainable synthesis of various 2-substituted pyridines and quinolines.

The present invention provides a process for the synthesis of pyrazine derivative comprises refluxing the reaction mixture of 1,2 amino alcohol and cobalt based catalyst of formula (I) in solvent at temperature in the range of 130 to 135° C. for the period in the range of 22 to 24 hrs to afford pyrazine derivative.

The process is carried out under argon atmosphere.

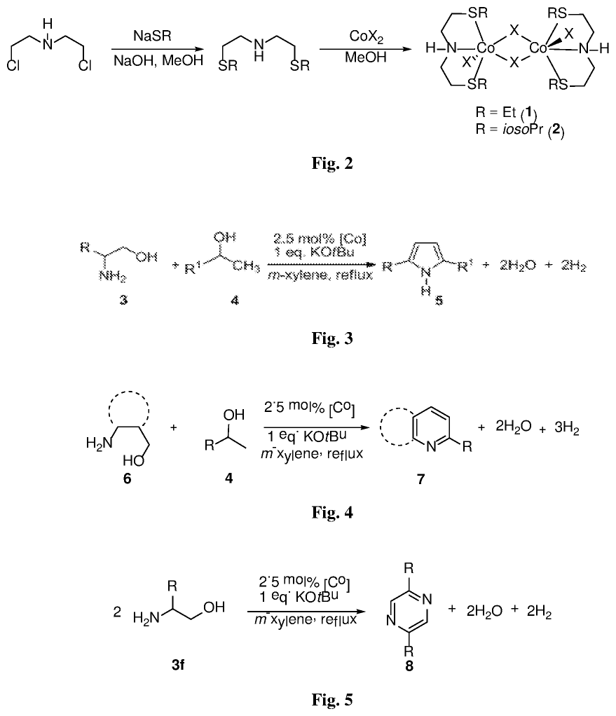

The present direct pyrazine synthesis catalyzed by phosphine free cobalt based catalyst of present invention is tested for gram-scale synthesis, and it worked excellently and gave 8 in 61% (1.02 g) isolated yield. [FIG. 5] This result implies that the phosphine free cobalt-based catalytic system of present invention has potential for the large-scale production of pyrazine under operationally simple, environmentally benign conditions.

### EXAMPLES

Following examples are given by way of illustration therefore should not be construed to limit the scope of the invention.

### Example 1: Synthesis of Ligands

### a) Bis(2-(ethylthio)ethyl)amine (^{Et}SNS; L1)

To a solution of bis(2-chloroethyl)amine hydrochloride (2.39 g, 13.4 mmol) in methanol (20 mL), 0.627 g of NaOH (15.7 mmol) and 2.5 g of sodium ethanethiolate (29.5 mmol) was added step wise. The resulting reaction mixture was allowed to stir for 12 h at 30° C., then the solvent was removed under reduced pressure, subsequently the reaction mixture was extracted with dichloromethane. The organic layer was collected and dried over anhyd. Na₂SO₄, then evaporated in vacuum under the reduced and the product (L1) was purified through neutral alumina column chromatography. Yield (0.862 g, 50%). ¹H NMR (500 MHz, CHLOROFORM-d) δ=2.83 (t, J=6.5 Hz, 4H), 2.69 (t, J=6.9 Hz, 4H), 2.55 (q, J=7.2 Hz, 4H), 1.98 (s, br, 1H), 1.26 (t, J=7.25 Hz, 6H). HRMS (EI): m/z Calcd for C₈H₁₉NS₂ [M+H]⁺: 194.0959; Found: 194.1043.

### b) Bis(2-(isopropylthio)ethyl)amine (^{iosPr}SNS; L2)

To a solution of bis(2-chloroethyl)amine hydrochloride (2.39 g, 13.4 mmol) in methanol (20 mL), 0.627 g of NaOH (15.7 mmol) and 2.9 g of sodium 2-propanethiolate (29.5 mmol) was added step wise. The resulting reaction mixture was allowed to stir for 12 h at 30° C., then the solvent was removed under reduced pressure, subsequently the reaction mixture was extracted with dichloromethane. The organic layer was collected and dried over anhyd.Na₂SO₄, then evaporated in vacuum under the reduced and the product (L2) was purified through neutral alumina column chromatography. Yield (1.42 g, 48%). ¹H NMR (500 MHz, CHLOROFORM-d) δ=2.94 (2H), 2.83 (t, J=6.9 Hz, 4H), 2.71 (t, J=6.5 Hz, 4H), 2.05 (s, br, 1H), 1.28 (d, J=6.5 Hz, 12H). ¹³C NMR (126 MHz, CHLOROFORM-d) δ =48.64, 34.81, 30.74, 23.49. HRMS (EI): m/z Calcd for C₁₀H₂₄NS₂ [M+H]⁺: 222.1345; Found: 222.1356.

### Example 2: Synthesis of Cobalt-Complexes

### a) Dimer of Co(II)chloride:bis(2-(diethyl-λ³-sulfanyl)ethyl)amine

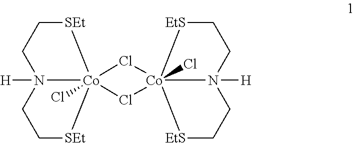

Anhydrous CoCl₂ (130 mg, 1 mmol) in methanol (2 mL) was added drop-wise to solution of ^{Et}SNS (L1) (193 mg, 1 mmol) in MeOH (2 mL) with stirring. The resulting reaction mixture was allowed to stir for 3 h at 30° C. The resulting solution was passed through syringe filter and dried in vacuo giving a blue crystalline powder. The crystal suitable for a single-crystal X-ray diffraction was obtained from MeOH: diethyl ether (by diffusion method) at 30° C. after one day.

Yield (249 mg, 77%); IR (KBr): 3213, 2936, 2868, 2792, 1625, 1462, 1412, 1377, 1306, 1268, 1093, 957, 731 cm⁻¹. The UV-Visible spectra of 1 recorded in acetonitrile show absorption centred at 589 and 680 nm. Elemental analysis calcd (%) for C₁₆H₃₈Cl₄Co₂N₂S₄: C 29.73; H 5.93; N 4.33; S 19.84; found: C 29.98; H 6.08; N 4.40; S 19.89. The formation of dimer is evidenced by MALDI-TOF mass spectrum (m/z=643.62). EPR study of 1 shows the paramagnetic nature of cobalt (II) complex and the g value is 2.58. Magnetic moment: 2.23 μB.

### b) Dimer of Co(II)chloride:bis(2-(isopropylthio)ethyl)amine

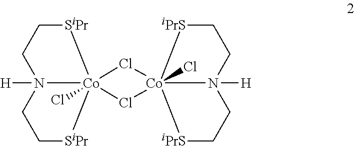

Anhydrous CoCl₂ (130 mg, 1 mmol) in methanol (2 mL) was added drop-wise to solution of L2 (221 mg, 1 mmol) in MeOH (2 mL) with stirring. The resulting reaction mixture was allowed to stir for 3 h at 30° C. The resulting solution was passed through syringe filter and then kept for crystallization (diffusion method using diethyl ether). After 1 day, blue crystalline solid was obtained.

Yield (252 mg, 72%). IR (KBr): 3236, 2959, 2867, 2808, 2751, 1626, 1524, 1449, 1368, 1248, 1155, 997, 955, 725 cm⁻¹. The UV-Visible spectra of 2 recorded in acetonitrile show absorption centred at 588 and 680 nm. EPR study of 2 shows the paramagnetic nature of cobalt (II) complexes and having the g, and g_{y} values 2.33 and 2.13, respectively, Elemental analysis calcd (%) for C₂₀H₄₆Cl₄Co₂N₂S₄: C 34.19; H 6.60; N 3.99; S 18.25; found: C 34.30; H 6.78; N 4.10; S 18.36. The formation of dimer is evidenced by MALDI-TOF mass spectrum (m/z=701.42). Magnetic moment: 2.29 μB.

### c) Dimer of Co(II)bromide:bis(2-(diethyl-λ³-sulfanyl)ethyl)amine

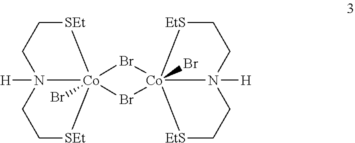

Anhydrous CoBr₂ (219 mg, 1 mmol) in methanol (2 mL) was added dropwise to solution of SNS-L1 (193 mg, 1 mmol) in MeOH (2 mL) with stirring. The resulting reaction mixture was allowed to stir for 3 h at 30° C. The resulting solution was passed through syringe filter and collected in small glass vail and kept for crystallization via diffusion method using diethyl ether as external solvent, which afford blue crystalline solid material. Yield (267 mg, 65%); IR (KBr): 3212, 2964, 2928, 2867, 2788, 1463, 1412, 1377, 1305, 1268, 1230, 1148, 1093, 1051, 958 cm⁻¹.

### d) Dimer of Co(II)bromide:bis(2-(isopropylthio)ethyl)amine

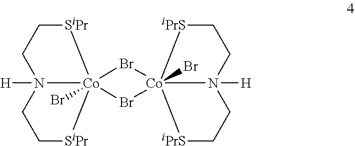

Anhydrous CoBr₂ (219 mg, 1 mmol) in methanol (2 mL) was added dropwise to solution of SNS-L2 (221 mg, 1 mmol) in MeOH (2 mL) with stirring. The resulting reaction mixture was allowed to stir for 3 h at 30° C. The resulting solution was passed through syringe filter and collected in small glass vail and kept for crystallization via diffusion method using diethyl ether as external solvent, which afford blue crystalline solid material. Yield (254 mg, 58%); IR (KBr): 3217, 2958, 2922, 2865, 1464, 1412, 1368, 1307, 1247, 1148, 1055, 960, 726 cm⁻¹. HRMS (EI) or ESI mass are tried several times but in all case under the mass condition ligand is coming out from the metal center.

### Example 3: Synthesis of Aromatic Heterocyclic of Formula (I) at Different Reaction Conditions

### a) Reaction with Different Solvent^{a}

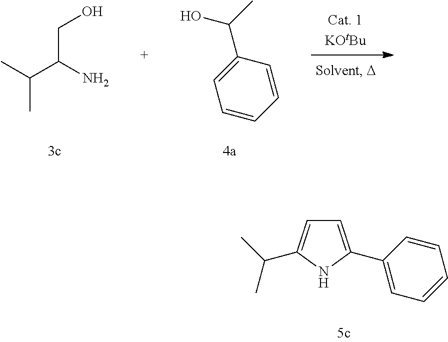

TABLE 1

| Entry | Solvent | Yield (%)^{b} |
|---|---|---|
| 1 | Toluene | 47 |
| 2 | m-xylene | 77 |
| 3 | Mesitylene | 57 |
| 4 | n-octane | 52 |
| 5 | THF | 32 |

^{a} Reactions performed using amino alcohol 3c (0.125 mmol), 1-Phenyl ethanol 4a (0.15 mmol), catalyst 1 (2.5 mol %), KO^{t}Bu (1.1 equiv.) at 180° C. of bath temp.
^{b}Yield determined by GC using 1,4-dibromo butane as an internal standard.

### b) Reaction with Different Catalyst^{a}

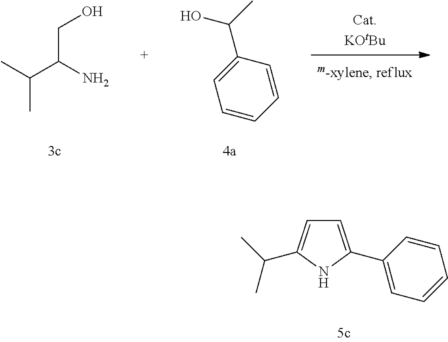

TABLE 2

| Entry | Catalyst | Yield (%)^{b} |
|---|---|---|
| 1 | Cat. 1 | 77 |
| 2 | Cat. 2 | 34 |
| 3 | CoCl₂ | trace |
| 4 | — | NR |
| 5 | RuCl₂(PPh₃)[HN(C₂H₄SEt)₂] | 45 |

^{a} Reactions performed using amino alcohol 3c (0.125 mmol), 1-Phenyl ethanol 4a (0.15 mmol), catalyst (2.5 mol %), KO^{t}Bu (1.1 equiv.) reflux at 150° C. to 180° C.
^{b}Yield determined by GC using 1,4-dibromo butane as an internal standard. NR = No reaction.

### c) Reaction with Different Base^{a}

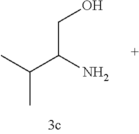

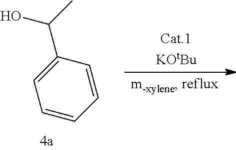

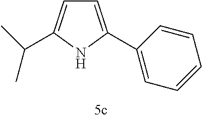

| Entry | Base | Yield (%)^{b} |
|---|---|---|
| 1 | NaO^{t}Bu | 63 |
| 2 | NaOⁱPr | 17 |
| 3 | KOH | 62 |
| 4 | KO^{t}Bu | 77 |
| 5 | KHMDS | 67 |
| 6 | KH | 71 |
| 7 | K₂CO₃ | NR |
| 8 | KOAc | NR |
| 9 | — | NR |

^{a}Reactions performed using amino alcohol 3c (0.125 mmol), 1-Phenyl ethanol 4a (0.15 mmol), catalyst 1 (2.5 mol %), base (1.1 equiv.) reflux at 150° C. to 180° C.
^{b}Yield determined by GC using 1,4-dibromo butane as an internal standard.
NR = No reaction.

### d) Reaction with Different Base Amount^{a}

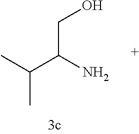

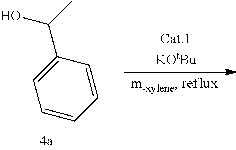

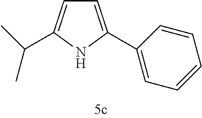

| Entry | Base (x equiv) | Yield (%)^{b} |
|---|---|---|
| 1 | 0.5 eq | 20 |
| 2 | 1.1 eq | 77 |
| 3 | 1.5 eq | 55 |
| 4 | 2 eq | 39 |
| 5 | 2.5 eq | 35 |

^{a}Reactions performed using amino alcohol 3c (0.125 mmol), 1-Phenyl ethanol 4a (0.15 mmol), catalyst (2.5 mol %), KO^{t}Bu (equiv.) reflux at 150° C. to 180° C.
^{b}Yield determined by GC using 1,4-dibromo butane as an internal standard.

### e) Reaction with Different Temperature^{a}

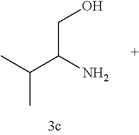

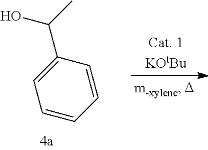

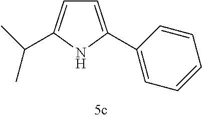

| Entry | Temperature | Yield (%)^{b} |
|---|---|---|
| 1 | 50° C. | NR |
| 2 | 80° C. | trace |
| 3 | 120° C. | 23 |
| 4 | 150° C. | 48 |
| 5 | 180° C. | 77 |
| | (reflux) | |

^{a}Reactions performed using amino alcohol 3c (0.125 mmol), 1-Phenyl ethanol 4a (0.15 mmol), catalyst (2.5 mol %), KO^{t}Bu (equiv.) at different (bath) temperature.
^{b}Yield determined by GC using 1,4-dibromo butane as an internal standard.
NR = No reaction.

### f) Reaction with Different Alcohol and Amino Alcohol Ratio^{a}

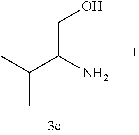

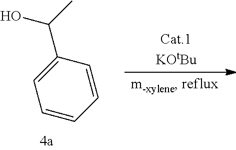

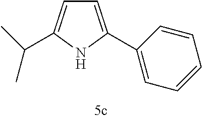

| | Alcohol/amino | |
|---|---|---|
| Entry | alcohol ratio | Yield (%)^{b} |
| 1 | 1/1 | 69 |
| 2 | 1/1 (cat 2) | 67 |
| 3 | 1.5/1 | 71 |
| 4 | 2/1 | 77 |
| 5 | 3/1 | 65 |

^{a}Reactions performed using amino alcohol 3c (0.125 mmol), 1-Phenyl ethanol 4a (0.15 mmol), catalyst (2.5 mol %), KO^{t}Bu (equiv.) reflux at 150° C. to 180° C.
^{b}Yield determined by GC using 1,4-dibromo butane as an internal standard.
NR = No reaction.

### Example 4: Synthesis of Aromatic Heterocyclic Compound of Formula (I)

### (a) General Procedure for the Synthesis of 1H-Pyrroles

To an oven-dried 15 mL ace pressure tube, 1,2 amino alcohol 3 (0.25 mmol), secondary alcohol 4 (0.5 mmol), Co-complex 1 (2.5 mol %) and m-xylene (2 mL) were added under a gentle stream of argon. The mixture was of heated at 150° C. to 180° C. for 24 h followed by cooling to room temperature. The reaction mixture was diluted with water (4 mL) and extracted with dichloromethane (3×5 mL). The resultant organic layer was dried over anhydrous Na₂SO₄ and the solvent was evaporated under reduced pressure. The crude mixture was purified by silica gel column chromatography (230-400 mesh size) using petroleum-ether/ethyl acetate as an eluting system.

### i. 2-methyl-5-phenyl-1H-pyrrole (5a)

To an oven-dried 15 mL ace pressure tube, 2-aminopropan-1-ol (0.25 mmol), 1-phenylethanol (0.5 mmol), Co-complex 1 (2.5 mol %) and m-xylene (2 mL) were added under a gentle stream of argon. The mixture was of heated at 150° C. to 180° C. for 24 h followed by cooling to room temperature. The reaction mixture was diluted with water (4 mL) and extracted with dichloromethane (3×5 mL). The resultant organic layer was dried over anhydrous Na₂SO₄ and the solvent was evaporated under reduced pressure. The crude mixture was purified by silica gel column chromatography (230-400 mesh size) using petroleum-ether/ethyl acetate as an eluting system.

Colorless oil. Yield: 77%. ¹H NMR (500 MHz, CHLOROFORM-d) δ=8.13 (s, br, 1H), 7.45 (d, J=7.2 Hz, 2H), 7.35 (t, J=7.2 Hz, 2H), 7.17 (t, J=7.2 Hz, 1H), 6.41 (s, 1H), 5.98 (s, 1H), 2.35 (s, 3H). ¹³C NMR (126 MHz, CHLOROFORM-d) δ=132.94, 129.02, 128.80, 125.64, 123.33, 107.93, 106.18, 13.19. HRMS (EI): m/z Calcd for C₁₁H₁₂N [M+H]⁺: 158.0964; Found: 158.0965.

### ii. 2-ethyl-5-phenyl-1H-pyrrole (5b)

Colorless oil. Yield: 81%. ¹H NMR (500 MHz, CHLOROFORM-d) δ=8.15 (s, br, 1H), 7.45 (d, J=8.0 Hz, 2H), 7.36 (t, J=7.2 Hz, 2H), 7.18 (t, J=7.2 Hz, 1H), 6.44 (s, 1H), 6.01 (s, 1H), 2.71 (q, J=7.6 Hz, 2H), 1.31 (t, J=7.6 Hz, 3H). ¹³C NMR (126 MHz, CHLOROFORM-d) δ=135.60, 132.99, 130.58, 128.79, 125.65, 123.38, 106.23, 105.98, 21.00, 13.59. HRMS (EI): m/z Calcd for C₁₂H₁₂N [M−H]⁺: 170.0964; Found: 170.0964.

### iii. 2-isopropyl-5-phenyl-1H-pyrrole (5c)

Colorless oil. Yield: 73%. ¹H NMR (500 MHz, CHLOROFORM-d) δ=8.14 (s, br, 1H), 7.45 (d, J=7.2 Hz, 2H), 7.35 (t, J=7.6 Hz, 2H), 7.17 (t, J=7.2 Hz, 1H), 6.42 (s, 1H), 6.00 (s, 1H), 3.1-2.98 (m, 1H), 1.32 (d, J=6.9 Hz, 6H). ¹³C NMR (126 MHz, CHLOROFORM-d) δ =140.30, 133.03, 130.46, 128.79, 125.67, 123.43, 105.81, 104.97, 27.21, 22.66. HRMS (EI): m/z Calcd for C₁₃H₁₆N [M+H]⁺: 186.1277; Found: 186.1279.

### iv. 2-isobutyl-5-phenyl-1H-pyrrole (5d)

Colorless oil. Yield: 86%. ¹H NMR (500 MHz, CHLOROFORM-d) δ=8.10 (s, br, 1H), 7.45 (d, J=7.6 Hz, 2H), 7.35 (t, J=7.6 Hz, 2H), 7.17 (t, J=7.2 Hz, 1H), 6.44 (s, 1H), 5.98 (s, 1H), 2.52 (d, J=7.2 Hz, 2H), 1.94-1.88 (m, 1H), 0.98 (d, J=6.9 Hz, 6H). ¹³C NMR (126 MHz, CHLOROFORM-d) δ=133.21, 133.00, 130.42, 128.79, 125.57, 123.30, 107.99, 106.04, 37.38, 29.27, 22.45. HRMS (EI): m/z Calcd for C₁₄H₁₆N [M−H]⁺: 198.1277; Found: 198.1277.

### v. 2-sec-butyl-5-phenyl-1H-pyrrole (5e)

Colorless oil. Yield: 78%. ¹H NMR (500 MHz, CHLOROFORM-d) δ=8.13 (s, br, 1H), 7.45 (d, J=7.6 Hz, 2H), 7.37 (t, J=7.6 Hz, 2H), 7.19 (t, J=7.2 Hz, 1H), 6.46 (s, 1H), 6.01 (s, 1H), 2.77-2.73 (m, 1H), 1.74-1.68 (m, 1H), 1.64-1.61 (m, 1H), 1.32 (d, J=6.9 Hz, 3H), 0.96 (t, J=7.2 Hz, 3H). ¹³C NMR (126 MHz, CHLOROFORM-d) δ=139.17, 133.05, 130.24, 128.77, 125.58, 123.34, 105.83, 105.64, 34.41, 30.23, 20.06, 11.82. HRMS (EI): m/z Calcd for C₁₄H₁₈N [M+H]⁺: 200.1434; Found: 200.1432.

### vi. 2,5-diphenyl-1H-pyrrole (5f)

Brown liquid. Yield: 58%. ¹H NMR (200 MHz, CHLOROFORM-d) δ=8.60 (s, br, 1H), 7.55 (d, J=7.7 Hz, 4H), 7.40 (t, J=7.3 Hz, 4H), 7.27 (t, J=7.3 Hz, 2H), 6.60 (d, J=2.5 Hz, 2H). HRMS (EI): m/z Calcd for C₁₆H₁₃N [M+H]⁺: 219.1043; Found: 219.1043. (Known compound: Michlik, S.; Kempe, R. Nat. Chem. 2013, 5, 140).

### vii. 2-benzyl-5-phenyl-1H-pyrrole (5g)

Light brown oil. Yield: 70%. ¹H NMR (500 MHz, CHLOROFORM-d) δ=8.05 (s, br, 1H), 7.40 (d, J=7.6 Hz, 2H), 7.35-7.31 (m, 5H), 7.26 (d, J=7.2 Hz, 2H), 7.16 (t, J=7.2 Hz, 1H), 6.44 (s, 1H), 6.06 (s, 1H), 4.04 (s, 2H). HRMS (EI): m/z Calcd for C₁₇H₁₄N [M−H]⁺: 232.1121; Found: 232.1121. (Known compound: Michlik, S.; Kempe, R. Nat. Chem. 2013, 5, 140).

### viii. 2-isopropyl-5-p-tolyl-1H-pyrrole (5h)

Colorless oil. Yield: 85%. ¹H NMR (500 MHz, CHLOROFORM-d) δ=8.10 (s, br, 1H), 7.36 (d, J=8.5 Hz, 2H), 7.17 (d, J=7.9 Hz, 2H), 6.38 (s, 1H), 5.99 (s, 1H), 3.00-2.98 (m, 1H), 2.36 (s, 3H), 1.32 (d, J=7.3 Hz, 6H). ¹³C NMR (126 MHz, CHLOROFORM-d) δ=139.84, 135.33, 130.60, 130.31, 129.45, 123.44, 105.18, 104.76, 27.19, 22.66, 21.06. HRMS (EI): m/z Calcd for C₁₄H₁₈N [M+H]⁺: 200.1434; Found: 200.1431.

### ix. 2-(4-chlorophenyl)-5-isopropyl-1H-pyrrole (5i)

Colorless oil. Yield: 70%. ¹H NMR (500 MHz, CHLOROFORM-d) δ=8.10 (s, br, 1H), 7.37 (d, J=8.4 Hz, 2H), 7.31 (d, J=8.4 Hz, 2H), 6.41 (s, 1H), 6.00 (s, 1H), 3.01-2.95 (m, 1H), 1.32 (d, J=6.9 Hz, 6H). ¹³C NMR (126 MHz, CHLOROFORM-d) δ=140.78, 131.52, 131.11, 128.92, 124.56, 123.43, 106.36, 105.23, 27.23, 22.64. HRMS (EI): m/z Calcd for C₁₃H₁₅ClN [M+H]⁺: 220.0888; Found: 220.0886.

### x. 2-isopropyl-5-(4-methoxyphenyl)-1H-pyrrole (5j)

White solid. Yield: 89%. ¹H NMR (500 MHz, CHLOROFORM-d) δ=8.03 (s, br, 1H), 7.38 (d, J=8.4 Hz, 2H), 6.91 (d, J=8.4 Hz, 2H), 6.30 (s, 1H), 5.97 (s, 1H), 3.83 (s, 3H), 3.00-2.95 (m, 1H), 1.32 (d, J=6.9 Hz, 6H). ¹³C NMR (126 MHz, CHLOROFORM-d) δ=157.91, 139.58, 130.48, 126.22, 124.89, 114.26, 104.66, 55.30, 27.18, 22.68. HRMS (EI): m/z Calcd for C₁₄H₁₈ON [M+H]⁺: 216.1383; Found: 216.1381.

### xi. 4-(5-isopropyl-1H-pyrrol-2-yl)aniline (5k)

Light brown liquid. Yield: 67%. ¹H NMR (500 MHz, CHLOROFORM-d) δ=7.99 (s, br, 1H), 7.26 (d, J=6.7 Hz, 2H), 6.69 (d, J=7.9 Hz, 2H), 6.24 (s, 1H), 5.95 (s, 1H), 3.65 (s, br, 3H), 2.99-2.94 (m, 1H), 1.31 (d, J=6.7 Hz, 6H). ¹³C NMR (126 MHz, CHLOROFORM-d) δ =144.48, 139.08, 131.03, 124.93, 124.31, 115.53, 104.45, 103.88, 27.16, 22.69. HRMS (EI): m/z Calcd for C₁₃H₁₇N₂ [M+H]⁺: 201.1386; Found: 201.1385.

### xii. 2-isopropyl-5-m-tolyl-1H-pyrrole (5l)

Colorless oil. Yield: 70%. ¹H NMR (500 MHz, CHLOROFORM-d) δ=8.15 (s, br, 1H), 7.76 (s, 1H), 7.28-7.26 (m, 2H), 7.03-7.01 (m, 1H), 6.42 (s, 1H), 6.01 (s, 1H), 3.03-2.97 (m, 1H), 2.40 (s, 3H), 1.34 (d, J=7.3 Hz, 6H). ¹³C NMR (126 MHz, CHLOROFORM-d) δ=140.12, 138.32, 133.69, 128.68, 128.40, 126.50, 124.18, 120.62, 105.67, 104.87, 27.21, 22.66, 21.52. HRMS (EI): m/z Calcd for C₁₄H₁₈N [M+H]⁺: 200.1434; Found: 200.1432.

### xiii. 2-isopropyl-5-(naphthalen-1-yl)-1H-pyrrole (5m)

Light yellow sticky liquid. Yield: 61%. ¹H NMR (500 MHz, CHLOROFORM-d) δ=8.39-8.38 (m, 1H), 8.16 (s, br, 1H), 7.90-7.89 (m, 1H), 7.80-7.79 (m, 1H), 7.51 (m, 4H), 6.44 (s, 1H), 6.12 (s, 1H), 3.11-3.03 (m, 1H), 3.37 (d, J=6.5 Hz, 6H). ¹³C NMR (126 MHz, CHLOROFORM-d) δ=139.82, 134.10, 131.84, 131.28, 128.89, 128.39, 127.05, 126.19, 125.85, 125.62, 125.45, 109.34, 104.34, 27.17, 22.67. HRMS (EI): m/z Calcd for C₁₇H₁₈N [M+H]⁺: 236.1434; Found: 236.1425.

### xiv. 2-isopropyl-5-octyl-1H-pyrrole (5n)

Ration of alcohol/amino alcohol=1.5/1 has taken under the identical reaction condition. Colorless oil. Yield: 45%. ¹H NMR (200 MHz, CHLOROFORM-d) δ=5.79 (d, J=2.3 Hz, 2H), 2.96-2.82 (m, 1H), 2.56 (t, J=7.3 Hz, 2H), 1.62 (m, 2H), 1.27 (m, 16H), 0.89 (t, J=5.0 Hz, 3H). HRMS (EI): m/z Calcd for C₁₅H₂₈N [M+H]⁺: 222.2216; Found: 222.2213. The product contains dehydrogenated product derived from secondary alcohol (Product: other dehydrogenated products=1:1.5).

### xv. 2-isobutyl-5-(naphthalen-2-yl)-1H-pyrrole (5o)

Light yellow sticky liquid. Yield: 74%. ¹H NMR (500 MHz, CHLOROFORM-d) δ=8.26 (s, br, 1H), 7.83-7.79 (m, 4H), 7.66 (d, J=8.8 Hz, 1H), 7.47 (t, J=7.2 Hz, 1H), 7.43 (t, J=7.2 Hz, 1H), 6.58 (s, 1H), 6.04 (s, 1H), 2.56 (d, J=6.9 Hz, 2H), 1.98-1.93 (m, 1H), 1.01 (d, J=6.5 Hz, 6H). ¹³C NMR (126 MHz, CHLOROFORM-d) δ=133.85, 131.81, 130.43, 128.45, 127.70, 127.51, 126.36, 125.06, 123.06, 120.02, 108.23, 106.84, 37.44, 29.29, 22.51. HRMS (EI): m/z Calcd for C1₈H₂₀N [M+H]⁺: 250.1590; Found: 250.1583.

### b) General Procedure for the Synthesis Pyridine Derivatives

To an oven-dried 15 mL ace pressure tube, 1,2 amino alcohol 6 (0.25 mmol), secondary alcohol 4 (0.5 mmol), Co-complex 1 (2.5 mol %) and m-xylene (1 mL) were added under a gentle stream of argon. The mixture was heated at 150° C. to 180° C. for 24 h followed by cooling to room temperature. The reaction mixture was diluted with water (4 mL) and extracted with dichloromethane (3×5 mL). The resultant organic layer was dried over anhydrous Na₂SO₄ and the solvent was evaporated under reduced pressure. The crude mixture was purified by silica gel column chromatography (230-400 mesh size) using petroleum-ether/ethyl acetate as an eluting system.

### i. 2-Phenyl Pyridine (7a)

To an oven-dried 15 mL ace pressure tube, (2-aminophenyl)methanol 6 (0.25 mmol), 1-phenylethan-1-ol 4 (0.5 mmol), Co-complex 1 (2.5 mol %) and m-xylene (1 mL) were added under a gentle stream of argon. The mixture was heated at 150° C. to 180° C. for 24 h followed by cooling to room temperature. The reaction mixture was diluted with water (4 mL) and extracted with dichloromethane (3×5 mL). The resultant organic layer was dried over anhydrous Na₂SO₄ and the solvent was evaporated under reduced pressure. The crude mixture was purified by silica gel column chromatography (230-400 mesh size) using petroleum-ether/ethyl acetate as an eluting system.

Colorless oil. Yield: 68%. ¹H NMR (500 MHz, CHLOROFORM-d) δ=8.71 (d, J=4.9 Hz, 1H), 8.01 (d, J=7.6 Hz, 2H), 7.73 (m, 2H), 7.49 (t, J=8.0 Hz, 2H), 7.43 (t, J=7.2 Hz, 1H), 7.23-7.21 (m, 1H). ¹³C NMR (126 MHz, CHLOROFORM-d) δ=157.35, 149.57, 139.31, 136.64, 128.86, 128.65, 126.81, 121.99, 120.45. HRMS (EI): m/z Calcd for C₁₁H₁₀N [M+H]⁺: 156.0808; Found: 156.0807.

### ii. 2-p-tolyl Pyridine (7b)

Colorless oil. Yield: 79%. ¹H NMR (500 MHz, CHLOROFORM-d) δ=8.69 (d, J=4.6 Hz, 1H), 7.90 (d, J=8.4 Hz, 2H), 7.76-7.71 (m, 2H), 7.29 (d, J=8.0 Hz, 2H), 7.21 (t, J=5.3 Hz, 1H), 2.42 (s, 3H). ¹³C NMR (126 MHz, CHLOROFORM-d) δ=157.49, 149.58, 138.93, 136.67, 129.46, 126.76, 121.78, 120.26, 21.25. HRMS (EI): m/z Calcd for C₁₂H₁₂N [M+H]⁺: 170.0964; Found: 170.0964.

### iii. 2-(4-methoxyphenyl) Pyridine (7c)

Colorless oil. Yield: 83%. ¹H NMR (500 MHz, CHLOROFORM-d) δ=8.66 (d, J=4.2 Hz, 1H), 7.96 (d, J=9.2 Hz, 2H), 7.72 (t, J=7.6 Hz, 1H), 7.69 (t, J=7.6 Hz, 1H), 7.18 (t, J=7.2 Hz, 1H), 7.01 (d, J=8.8 Hz, 1H), 3.88 (s, 3H). ¹³C NMR (126 MHz, CHLOROFORM-d) δ=160.46, 157.13, 149.54, 136.64, 132.04, 128.15, 121.39, 119.80, 114.11, 55.35. HRMS (EI): m/z Calcd for C₁₂H₁₂ON [M+H]⁺: 186.0913; Found: 186.0912.

### iv. 2-m-tolylpyridine (7d)

Colorless oil. Yield: 69%. ¹H NMR (500 MHz, CHLOROFORM-d) δ=8.70 (d, J=4.6 Hz, 1H), 7.85 (s, 1H), 7.77-7.72 (m, 3H), 7.38 (t, J=7.6 Hz, 1H), 7.24 (t, J=7.2 Hz, 2H), 2.45 (s, 3H). ¹³C NMR (126 MHz, CHLOROFORM-d) δ=157.65, 149.60, 139.35, 138.43, 136.70, 129.71, 128.63, 127.65, 123.99, 122.01, 120.64, 21.51. HRMS (EI): m/z Calcd for C₁₂H₁₂N [M+H]⁺: 170.0964; Found: 194.1043.

### v. 2-octyl Pyridine (7e)

Ration of alcohol/amino alcohol=1.5/1 has taken under the identical reaction condition.

Colorless oil. Yield: 60%. ¹H NMR (200 MHz, CHLOROFORM-d) δ=8.53 (d, J=4.8 Hz, 1H), 7.59 (t, J=9.3 Hz, 1H), 7.16-7.07 (m, 2H), 2.79 (t, J=8.1 Hz, 2H), 1.73 (m, 2H), 1.28 (m, 10H), 0.89 (t, J=6.7 Hz, 3H). HRMS (EI): m/z Calcd for C₁₃H₂₂N [M+H]⁺: 192.1747; Found: 192.1744. The product contains dehydrogenated product derived from secondary alcohol (Product: other dehydrogenated products=1:1.8). (Known compound: Nakamura, Y.; Yoshikai, N.; lies, L.; Nakamura, E. Org. Lett. 2012, 14, 12).

### vi. 2-decyl Pyridine (7f)

Ration of alcohol/amino alcohol=1.5/1 has taken under the identical reaction condition. Colorless oil. Yield: 63%. ¹H NMR (500 MHz, CHLOROFORM-d) δ=8.53 (d, J=4.6 Hz, 1H), 7.58 (t, J=7.6 Hz, 1H), 7.14 (d, J=7.6 Hz, 1H), 7.08 (t, J=5.7 Hz, 1H), 2.78 (t, J=7.6 Hz, 2H), 1.76-1.68 (m, 2H), 1.26 (m, 14H), 0.88 (t, J=6.9 Hz, 3H). HRMS (EI): m/z Calcd for C₁₅H₂₆N [M+H]⁺: 220.2060; Found: 220.2057. Unable to isolate complete pure product, product identified from its unreacted secondary alcohol. Product: unreacted secondary alcohol=1:1.6. (Known compound: Vandromme, L.; ReiBig, H.-U.; Groper, S.; Rabe, J. P. Eur. J. Org. Chem. 2008, 2049-2055).

### vii. 2-phenyl Quinoline (7g)

White solid. Yield: 81%. ¹H NMR (500 MHz, CHLOROFORM-d) δ=8.21 (t, J=9.2 Hz, 4H), 7.88 (d, J=8.4 Hz, 1H), 7.83 (d, J=8.0 Hz, 1H), 7.75 (t, J=8.0 Hz, 1H), 7.57-7.53 (m, 3H), 7.49 (t, J=7.2 Hz, 1H). ¹³C NMR (126 MHz, CHLOROFORM-d) δ=157.29, 148.24, 139.64, 136.70, 129.70, 129.59, 129.26, 128.79, 127.52, 127.41, 127.13, 126.22, 118.94. (Known compound: Rao, M. L. N.; Dhanorkar, R. J. Eur. J. Org. Chem. 2014, 5214-5228).

### viii. 2-(3-methoxyphenyl) Quinoline (7h)

Colorless oil. Yield: 75%. ¹H NMR (200 MHz, CHLOROFORM-d) δ=8.22-8.19 (m, 2H), 7.87-7.80 (m, 3H), 7.74 (t, J=8.5 Hz, 2H), 7.54 (t, J=7.3 Hz, 1H), 7.45 (t, J=7.9 Hz, 1H), 7.04 (d, J=8.5 Hz, 1H), 3.94 (s, 3H). ¹³C NMR (126 MHz, CHLOROFORM-d) δ=160.08, 157.04, 148.15, 141.09, 136.68, 129.74, 129.68, 129.58, 127.39, 126.25, 119.95, 119.02, 115.30, 112.66, 55.34. HRMS (EI): m/z Calcd for C₁₆H₁₄ON [M+H]⁺: 236.1070; Found: 236.1068.

### ix. 2-(4-fluorophenyl)quinoline (7i)

White solid. Yield: 72%. ¹H NMR (400 MHz, CHLOROFORM-d) δ=8.21-8.15 (m, 4H), 7.82 (d, J=8.5 Hz, 2H), 7.74 (t, J=6.7 Hz, 1H), 7.54 (t, J=7.3 Hz, 1H), 7.22 (t, J=8.5 Hz, 2H). ¹³C NMR (126 MHz, CHLOROFORM-d) δ=164.76, 162.77, 156.77, 148.19, 136.85, 136.85, 135.78, 129.74, 129.39, 129.33, 127.43, 127.04, 126.30, 118.58, 115.80, 115.63. HRMS (EI): m/z Calcd for C₁₅H₁₁NF [M+H]⁺: 224.0870; Found: 224.0869.

### x. 2-(4-(trifluoromethyl)phenyl)quinoline (7j)

White solid. Yield: 67%. ¹H NMR (500 MHz, CHLOROFORM-d) δ=8.29 (d, J=8.4 Hz, 2H), 8.25 (d, J=8.4 Hz, 1H), 8.20 (d, J=8.4 Hz, 1H), 7.88 (d, J=8.8 Hz, 1H), 7.85 (d, J=8.0 Hz, 1H), 7.80-7.75 (m, 3H), 7.58 (t, J=8.0 Hz, 1H). ¹³C NMR (126 MHz, CHLOROFORM-d) δ=155.62, 148.25, 142.92, 137.08, 129.96, 129.83, 127.80, 127.50, 126.82, 125.72, 125.69, 118.73. HRMS (EI): m/z Calcd for C₁₆H₁₁NF₃ [M+H]⁺: 274.0838; Found: 274.0838.

### xi. 2-(naphthalen-2-yl)quinoline (7k)

White solid. Yield: 87%. ¹H NMR (500 MHz, CHLOROFORM-d) δ=8.64 (s, 1H), 8.40 (d, J=8.8 Hz, 1H), 8.25 (d, J=8.4 Hz, 1H), 8.05-8.01 (m, 3H), 7.93-7.91 (m, 1H), 7.85 (d, J=8.0 Hz, 1H), 7.77 (t, J=6.9 Hz, 1H), 7.57-7.54 (m, 3H). ¹³C NMR (126 MHz, CHLOROFORM-d) δ=157.12, 148.35, 136.94, 136.76, 133.84, 133.48, 129.72, 129.68, 128.79, 128.54, 127.70, 127.46, 127.19, 127.11, 126.67, 126.30, 125.03, 119.11. HRMS (EI): m/z Calcd for C₁₉H₁₄N [M+H]⁺: 256.1121; Found: 256.1120.

### Example 5: General Procedure for the Synthesis of Pyrazine Derivative

To an oven-dried 15 mL ace pressure tube, 1,2 amino alcohol 3f (0.25 mmol), Co-complex 1 (2.5 mol %) and m-xylene (1 mL) were added under a gentle stream of argon. The mixture was heated at 135° C. (bath temperature). After 24 h, the reaction mixture was diluted with water (4 mL) and extracted with dichloromethane (3×5 mL). The resultant organic layer was dried over anhydrous Na₂SO₄ and the solvent was evaporated under reduced pressure. The crude mixture was purified by silica gel column chromatography (230-400 mesh size) using petroleum-ether/ethyl acetate as an eluting system.

Gram-scale synthesis: The present cobalt-catalyzed direct pyrazine synthesis was tested for the gram-scale synthesis, and it worked excellently and gave 8 in 61% (1.02 g) isolated yield.

### a. 2,5-Diphenyl Pyrazine (8)

To an oven-dried 15 mL ace pressure tube, 2-amino-2-phenylethan-1-ol 3f (0.25 mmol), Co-complex 1 (2.5 mol %) and m-xylene (1 mL) were added under a gentle stream of argon. The mixture was heated at 135° C. (bath temperature). After 24 h, the reaction mixture was diluted with water (4 mL) and extracted with dichloromethane (3×5 mL). The resultant organic layer was dried over anhydrous Na₂SO₄ and the solvent was evaporated under reduced pressure. The crude mixture was purified by silica gel column chromatography (230-400 mesh size) using petroleum-ether/ethyl acetate as an eluting system.

White solid. Yield: 68%. ¹H NMR (500 MHz, CHLOROFORM-d) δ=9.10 (s, 2H), 8.08 (d, J=7.2 Hz, 4H), 7.55 (t, J=7.2 Hz, 4H), 7.50 (q, J=7.2 Hz, 2H). ¹³C NMR (126 MHz, CHLOROFORM-d) δ=150.68, 141.25, 136.27, 129.77, 129.07, 126.79. (Known compound: Gnanaprakasam, B.; Balaraman, E.; Ben-David, Y.; Milstein, D. Angew. Chem. Int. Ed. 2011, 50, 12240).

### Advantages of the Invention

Use of a new, air-stable molecularly defined SNS-cobalt (II) complex.
This tandem annulation reaction operates under mild, eco-benign conditions with the liberation of hydrogen gas and water as the sole by-products.
Excellent step-economy and high atom-efficiency.
A simple, phosphine ligand-free Co (II)-complex as a precatalyst for the preparation of diverse N-heterocycles via dehydrogenative annulation of unprotected β-aminoalcohols with secondary alcohols.
Cobalt (II)-complexes are air-stable and their synthesis has the practical advantages of being straightforward, conveniently performed in open air atmosphere, and can be scaled up.

1-15. (canceled)

16. A phosphine free cobalt based catalyst of formula (I) 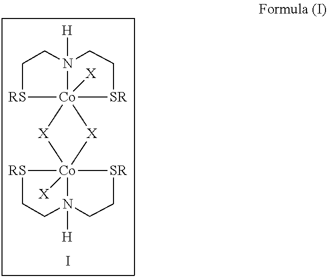
wherein:
R is selected from the group consisting of hydrogen, linear or branched alkyl, substituted or unsubstituted aryl and heteroaryl containing O, N atoms; and
X is selected from the group consisting of F, Cl, Br and I.

17. The phosphine free cobalt based catalyst of formula (I) as claimed in claim 16, wherein said cobalt based catalyst of formula (I) is selected from cobalt based dimer complex of bis(2-(diethyl-λ3-sulfanyl)ethyl)amine, bis(2-(isopropylthio)ethyl)amine, bis(2-(phenylthio)ethyl)amine or bis(2-((substituted)phenylthio)ethyl)amine.

18. A process for the preparation of cobalt based catalyst of formula (I) as claimed in claim 16, comprising the steps of:
i. preparing a solution of CoX₂ in solvent
ii. preparing a solution of SNS ligand in solvent
iii. mixing the solution of step (i) and (ii)
iv. stirring the reaction mixture of step (iii) at a temperature ranging from 25° C. to 30° C. for a time period ranging from 3 to 4 hours to yield cobalt based catalyst of formula (I).

19. The process as claimed in claim 18, wherein said CoX₂ is selected from the group consisting of Cobalt (II) chloride (CoCl₂), Cobalt (II) bromide (CoBr₂) or Cobalt (II) Iodide (CoI₂).

20. The process as claimed in claim 18, wherein said SNS ligand is selected from bis(2-(diethyl-λ3-sulfanyl)ethyl)amine (^{Et}SNS; L1) or bis(2-(isopropylthio)ethyl)amine (^{isoPr}SNS; L2).

21. The process as claimed in claim 18, wherein said solvent is selected from the group consisting of methanol, ethanol, tetrahydrofuran, acetonitrile or diethylether.

22. A process for the synthesis of aromatic heterocyclic compound of formula (II) 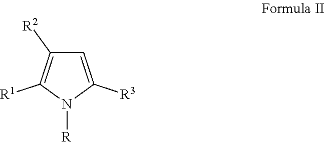
wherein:
n is selected from 0 or 1,
R is selected from the group consisting of hydrogen, linear or branched alkyl, substituted or unsubstituted aryl and heteroaryl containing 0, N atoms,
R¹, R², and R³ are same or different and independently selected from the group consisting of hydrogen, substituted or unsubstituted linear or branched alkyl, substituted or unsubstituted aryl,
R¹ and R² may form a substituted or unsubstituted cyclic or heterocyclic ring, and
the process comprises heating a reaction mixture of amino alcohol, alcohol, phosphine free cobalt based catalyst of formula (I) and base in a ratio ranging between 1:2:0.2:1 to 1:0.5:0.25:1.5 and solvent at a temperature ranging from 150 to 180° C. for a time period ranging from 24 to 30 hours followed by cooling the reaction mixture to afford aromatic heterocyclic compound of formula (II).

23. The process as claimed in claim 22, wherein said alcohol is selected from the group consisting of aliphatic short- and long-range primary alcohols, secondary alcohols, aromatic substituted or unsubstituted primary and secondary alcohols, heteroaromatic alcohols or cyclic alcohols.

24. The process as claimed in claim 22, wherein said alcohol is selected from the group consisting of 1-phenylethanol, 1-p-tolylethanol, 1-(4-chlorophenyl)ethanol, 1-(4-methoxyphenyl)ethanol, 1-(4-aminophenyl)ethanol, 1-(naphthalen-2-yl)ethanol, 1-(naphthalen-1-yl)ethanol, 2-decanol, 1-m-tolylethanol, 2-dodecanol, 1-(4-(trifluoromethyl)phenyl)ethanol and 1-(3-methoxyphenyl)ethanol.

25. The process as claimed in claim 22, wherein said amino alcohol is selected from aliphatic and aromatic β and γ amino alcohols.

26. The process as claimed in claim 22, wherein said amino alcohol is selected from the group consisting of 2-aminobutan-1-ol, 2-amino-3-methylbutan-1-ol, 2-amino-4-methylpentan-1-ol, 2-amino-3-methylpentan-1-ol, 2-amino-3-phenylpropan-1-ol, 2-amino-2-phenylethanol, 3-aminopropan-1-ol and (2-aminophenyl)methanol.

27. The process as claimed in claim 22, wherein said base is selected from the group consisting of potasium tert-butoxide (t-BuOK), sodium tert-butoxide (t-BuONa), lithium tert-butoxide (t-BuOLi), potassium hydride (KH), sodium hydride (NaH), potassium Bis (trimethylsilyl) amide [KHMDS], lithium bis (trimethylsilyl) amide [LiHMDS], sodium isopropoxide (NaOiPr), sodium ethoxide (NaOEt) or sodium methoxide (NaOMe).

28. The process as claimed in claim 22, wherein said solvent is selected from the group consisting of m-xylene, toluene, octane, mesitylene or decane.

29. The process as claimed in claim 22, wherein said aromatic heterocyclic compound of formula (II) is selected from the group consisting of
i. 2-methyl-5-phenyl-1H-pyrrole (5a),
ii. 2-ethyl-5-phenyl-1H-pyrrole (5b),
iii. 2-isopropyl-5-phenyl-1H-pyrrole (5c),
iv. 2-isobutyl-5-phenyl-1H-pyrrole (5d),
v. 2-sec-butyl-5-phenyl-1H-pyrrole (5e),
vi. 2,5-diphenyl-1H-pyrrole (5f),
vii. 2-benzyl-5-phenyl-1H-pyrrole (5g),
viii. 2-isopropyl-5-p-tolyl-1H-pyrrole (5h),
ix. 2-(4-chlorophenyl)-5-isopropyl-1H-pyrrole (5i),
x. 2-isopropyl-5-(4-methoxyphenyl)-1H-pyrrole (5j),
xi. 4-(5-isopropyl-1H-pyrrol-2-yl)aniline (5k),
xii. 2-isopropyl-5-m-tolyl-1H-pyrrole (5l),
xiii. 2-isopropyl-5-(naphthalen-1-yl)-1H-pyrrole (5m),
xiv. 2-isopropyl-5-octyl-1H-pyrrole (5n),
xv. 2-isobutyl-5-(naphthalen-2-yl)-1H-pyrrole (5o),
xvi. 2-phenyl pyridine (7a),
xvii. 2-p-tolylpyridine (7b),
xviii. 2-(4-methoxyphenyl) pyridine (7c),
xix. 2-m-tolylpyridine (7d),
xx. 2-octyl pyridine (7e),
xxi. 2-decyl pyridine (7f),
xxii. 2-phenyl quinoline (7g),
xxiii. 2-(3-methoxyphenyl) quinoline (7h),
xxiv. 2-(4-fluorophenyl)quinoline (7i),
xxv. 2-(4-(trifluoromethyl)phenyl)quinoline (7j) or
xxvi. 2-(naphthalen-2-yl)quinoline (7k).

30. A process for the synthesis of 2.5 di phenyl pyrazine comprising refluxing the reaction mixture of 2-amino-2-phenylethan-1-ol and cobalt based catalyst of formula (I) as claimed in claim 16 in solvent at temperature in the range of 130 to 135° C. for the period in the range of 22 to 24 hrs under argon atmosphere to afford the 2.5 di phenyl pyrazine.
